(19) United States
(12) Patent Application Publication
(10) Pub. No.: US 2012/0004309 A1
(43) Pub. Date: Jan. 5, 2012

(54) Hydroxamic Acid Derivatives, Preparation and Therapeutic Uses Thereof

(76) Inventors: Amin Khan, Chicago, IL (US); Paul Wood, Saskatoon (CA); Joseph Moskal, Evanston, IL (US)

(21) Appl. No.: 13/118,879

(22) Filed: May 31, 2011

Related U.S. Application Data
(63) Continuation of application No. PCT/US2009/066536, filed on Dec. 3, 2009.
(60) Provisional application No. 61/119,514, filed on Dec. 3, 2008.

(51) Int. Cl. A61K 31/185; A61P 25/28; A61P 25/00; C07C 259/06; A61P 25/16

(52) U.S. Cl. 514/575; 562/623

(57) ABSTRACT

Disclosed are amino alkyl/aryl hydroxamic acid compounds and pharmaceutical compositions containing such compounds. The disclosed compositions are useful as therapeutics for degenerative diseases in mammal.

### RELATED APPLICATION

This application is a continuation of PCT/US2009/066536 filed Dec. 3, 2009, which claims priority to U.S. Ser. No. 61/119,514, filed Dec. 3, 2008, which is hereby incorporated by reference in its entirety.

### BACKGROUND

The translation of degenerative disease mechanisms into effective therapeutics has been meager and disappointing. While the concept of “oxidative stress” has been implicated in the field of cellular degeneration, the concept is vague and has failed to differentiate critical events from epiphenomena and sequelae.

A corollary of current concepts of “oxidative stress” is the concept of “aldehyde load.” This concept relates to the production of reactive aldehydes which covalently modify proteins, nucleic acids, lipids and carbohydrates and activate apoptotic and necrotic pathways. There is increasing evidence that there are a number of metabolic pools that generate aldehydes in biologic systems. Major sources of reactive aldehydes in vivo include lipid peroxidation, glycation, amino acid oxidation and polyamine metabolism. Although the types of aldehydes produced by these processes are varied, the relevant aldehydes that are capable of exerting biological effects on the pathobiology of oxidant injury include 2-alkenals, 4-hydroxy-2-alkenals, ketoaldehydes and aminoaldehydes. Increased formation of reactive aldehydes and accumulation of aldehydes bound to proteins occurs in nearly every degenerative disease.

The toxicity of reactive aldehydes can result from a number of actions. Cytotoxicity with 2-alkenals (e.g. acrolein), 4-hydroxy-2-alkenals (e.g. 4-hydroxy-nonenal) and ketoaldehydes (e.g. malondialdehyde) involves activation of the intrinsic apoptotic cascade, independent of lysosomes. These aldehydes form covalent linkages with amino acids, proteins, nucleic acids and lipids, actions that can result in direct mitochondrial toxicity. Aminoaldehydes also have the potential for these toxic actions, but their lysosomotropic actions appear to be more important. Robust insults to cells with 3-aminopropanal, for example, can result in lysosomal rupture and cellular necrosis while lesser insults may result in lysosomal leakage of proteases that compromise mitochondrial integrity and thereby activate the intrinsic apoptotic cascade.

Increases in the levels of free aldehydes and protein bound aldehydes occur in neurodegenerative diseases such as Alzheimer's disease, Parkinson's disease, amyotrophic lateral sclerosis, Huntington's disease, multiple sclerosis, traumatic brain injury and brain ischemia-reperfusion injury. Aldehyde levels and protein-bound aldehyde are also elevated in age-related macular degeneration, myocardial infarction, renal ischemia-reperfusion injury, type II diabetes liver cirrhosis, and rheumatoid arthritis.

Recent progress in hydroxamic acid chemistry has facilitated the isolation of several naturally occurring, and the synthesis of a number of medicinally active, hydroxylamine derivatives. The structures of hydroxamic acids have been established along with their many biological activities. A series of o-, m-, and p-alkoxybenzo hydroxamic acids, for example, are highly effective against pathogenic fungi, while salicohydroxamic acids are effective antibacterial and antifungal agents. Beta-alkylaminopropiono-hydroxamic acids have hypotensive properties while other hydroxamic acids possess hypocholesteremic activity.

Since reactive aldehydes can be produced via multiple pathways resulting in a diverse array of reactive aldehyde products there is a need for cytoprotective agents that can chemically neutralize reactive aldehydes.

### SUMMARY

The present disclosure relates to the prevention of and treatment of degenerative diseases characterized by the reduction of a specific cell population by the excessive production of reactive aldehydes.

The present disclosure relates in part to hydroxamic acids, hydroxylamines, and mercapto agents which can act as aldehyde trapping agents and as cytoprotectants against aminoaldehydes and alkenal aldehydes in degenerative diseases.

For example, provided herein are compounds represented by formula Ib:

A compound of Formula Ib:

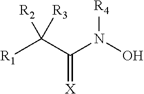

or a pharmaceutically acceptable salt thereof, wherein:

R₁ is selected from the group consisting of C₁₋₆alkyl, C₁₋₆substituted alkyl, C₂₋₆alkenyl, C₂₋₆substituted alkenyl, C₂₋₆alkynyl, C₂₋₆substituted alkynyl, C₃₋₆cycloalkyl, C₃₋₆substituted cycloalkyl, phenyl, cyano, hydroxyl, thiol, sulfonamide, amine,

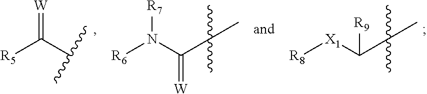

X is oxygen or sulfur;

X₁ is O, S, —S(O)— or —S(O)₂—;

W is oxygen or sulfur;

R₅ is selected from the group consisting of alkoxy, alkyl, substituted alkyl, alkenyl, substituted alkenyl, alkynyl, substituted alkynyl, cycloalkyl, substituted cycloalkyl, cycloalkenyl and substituted cycloalkenyl;

R₆ and R₇ are each independently selected from the group consisting of hydrogen, C₁₋₆alkyl, C₁₋₆substituted alkyl, C₂₋₆alkenyl, C₂₋₆substituted alkenyl, C₂₋₆alkynyl, C₂₋₆substituted alkynyl, C₃₋₆cycloalkyl, C₃₋₆substituted cycloalkyl; or R₆ and R₇ are joined to form an C₃₋₁₀cycloalkyl;

R₈ is selected from the group consisting of hydrogen, C₁₋₆alkyl, C₁₋₆substituted alkyl, C₂₋₆alkenyl, C₂₋₆substituted alkenyl, C₂₋₆alkynyl, C₂₋₆substituted alkynyl, C₃₋₆cycloalkyl, C₃₋₆ substituted cycloalkyl; and

R₉ is selected from the group consisting of hydrogen, C₁₋₆alkyl, C₁₋₆substituted alkyl, C₂₋₆alkenyl, C₂₋₆substituted alkenyl, C₂₋₆alkynyl, C₂₋₆substituted alkynyl, C₃₋₆cycloalkyl, C₃₋₆ substituted cycloalkyl;

R₂ is selected from the group consisting of hydrogen and C₁₋₆ alkyl;

R₃ is selected from the group consisting of C₁₋₆alkyl-NH—, NH₂—, -alkyl-C(O)—NH—, C₆H₅SO₂NH—, (C₆H₅SO₂)₂N—, C₄H₈N—, and C₅H₁₁NN—; and

R₄ is selected from the group consisting of hydrogen, C₁₋₆alkyl, C₁₋₆substituted alkyl.

Representative compounds provided by this disclosure include 2-amino-N-hydroxy-4-methylpentamide; 2-acetoamido-N-hydroxy-4-methylpentamide; 2-amino-N-hydroxypentamide; 3-amino-N-hydroxy-4-methylpentamide; 2-amino-N-hydroxypropanamide; 2-amino-N-hydroxybutanamide; 2-amino-N-hydroxy-3-methylpentamide; 2-amino-N-hydroxy-4-methylpentamide, and pharmaceutically acceptable salts thereof, for example trifluoroacetate salt (TFA).

Another embodiment of the present disclosure provides a method of treating or preventing a degenerative disease in a mammal. The method includes administering to the mammal a therapeutically effective amount of a pharmaceutical composition comprising the compound of Formula Ia or Ib.

A further embodiment of the present disclosure provides a method of decreasing cell death in a mammal. The method includes administering to the mammal a therapeutically effective amount of a pharmaceutical composition comprising the compound of Formula Ia or Ib.

In an embodiment, the degenerative disease includes at least one of multiple sclerosis, Parkinson's disease, Alzheimer's disease, amyotrophic lateral sclerosis, Huntington's disease, and traumatic brain injury, ischemia-reperfusion injury (stroke, renal, hepatic, myocardial infarction and transplantation), ocular degeneration (age-related macular degeneration), joint degeneration (rheumatoid arthritis), liver cirrhosis, and diabetes involving β-cell destruction.

Additional features and advantages are described herein, and will be apparent from, the following Detailed Description and the figures.

### BRIEF DESCRIPTION OF THE FIGURES

FIG. 1 is a graphical representation of median LDH levels over a 24-hour period in retinal cells from the retinal cell line E1A-NR.3 in controls (Con), cells treated with 400 μM 3-aminopropanal (3-AP) and co-treatment of cells with 2-amino-N-hydroxyl-methylpentamide (AK-10) at 150 μM, 300 μM, and 600 μM. Mean±SEM (N=8).

FIG. 2 is a graphical representation of median LDH levels over a 24-hour period in retinal cells from the retinal cell line E1A-NR.3 in controls (Con), cells treated with 400 μM 3-AP and delayed co-treatment of cells with 300 μM AK-10 at 0.5 hour, 1 hour, 1.5 hours, 2 hours, 2.5 hours and 3 hours. Mean±SEM (N=8).

FIG. 3 is a graphical representation of glutamate levels in hippocampal slices of Male Sprague Dawley rats (200 g; Harlan) before, during and after incubation for 5 minutes in KCl with no treatment (Control), treatment with trimethyltin (TMT) alone and treatment with TMT and AK-10.

### DETAILED DESCRIPTION

The present disclosure provides compositions and methods effective in sequestering cytotoxic aldehydes as a therapeutic means for treating degenerative diseases.

The present disclosure relates in part to bifunctional/multifunctional amino hydroxamic acids as therapeutics which are useful to sequester toxic aldehyde products of processes such as oxidative stress, intermediary metabolism, polyamine metabolism and myeloperoxidase activity. In particular, the compounds of the present disclosure may be useful for preventing and treating degenerative diseases without producing undesired side effects. Since reactive aldehydes are produced by diverse pathways, aldehyde-sequestering agents disclosed herein are optimal drug candidates to safely remove these cytotoxic metabolites. In particular, the present disclosure provides amino hydroxamic acid derivatives of the amino acid leucine and their use as therapeutic agents.

It has been surprisingly found that among other properties, the amino alkyl/aryl hydroxamic acid of the present disclosure sequester cytotoxic aldehydes such as 3-aminopropanal and acrolein and/are effective in various in vivo models. Accordingly, the compounds and pharmaceutical compositions of the present disclosure are effective therapeutics for treating degenerative diseases in mammals including humans.

Without being bound to any particular theory, among other properties, the amino alkyl/aryl amino hydroxamic acid compounds of this invention are believed to inhibit cell death by neutralizing the effects of aldehydes generated during oxidative stress, increased polyamine metabolism and aberrant intermediary metabolism. Compounds having such properties are useful for treating neurodegeneration (multiple sclerosis, Parkinson's disease, Alzheimer's disease, amyotrophic lateral sclerosis, Huntington's disease, and traumatic brain injury, ischemia-reperfusion injury, stroke, renal, hepatic, myocardial infarction and transplantation, ocular degeneration (age-related macular degeneration), joint degeneration, rheumatoid arthritis, liver cirrhosis, and diabetes involving β-cell destruction.

The multifunctional compounds may include a chemical moiety that can function as an antioxidant component, preferably without affecting the stability and action of the terminal group such as pro-drugs. Examples include: ether, ester, amide and nitric oxide-donor.

Accordingly, in an embodiment, a composition includes compounds having the structural formula set forth in Formula Ia:

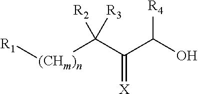

or a pharmaceutically acceptable salt thereof, where m may be an integer with a value ranging from zero to two; n may be an integer with a value ranging from one to six; R₂ and R₃ may include an amino group, a small alkyl, or a halide. One of either R₂ or R₃ may include an amino group and the other a small alkyl such as methyl, ethyl propyl, or halogen group such as fluoro, chloro and bromo. R₄ may include a hydrogen, small alkyl, substituted alkyl; X may include an oxygen or sulfur; R₁ and R₂ may include a hydrogen, alkyl, substituted alkyl, alkenyl, substituted alkenyl, alkynyl, substituted alkynyl, cycloalkyl, substituted cycloalkyl, cycloalkenyl, substituted cycloalkenyl, phenyl, substituted phenyl, heterocyclic, halide, nitrate, nitrite, nitrile, hydroxyl, thiol, sulfonamide, amine, guanidine, isoguanidine, cyanate, isocyanate, and carboxylate, or one of the following structural formulae:

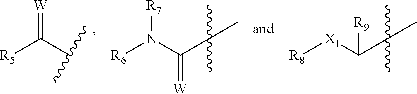

where X may be oxygen, sulfur, —S(O)— or —S(O)₂—, ═NH, ═NCN, X₁ is O, S, —S(O)— or —S(O)₂—;

W may be oxygen, sulfur, or pharmaceutically-acceptable salts thereof; R₅ may include an alkoxy, alkyl, substituted alkyl, alkenyl, substituted alkenyl, alkynyl, substituted alkynyl, cycloalkyl, substituted cycloalkyl, cycloalkenyl or substituted cycloalkenyl; R₆ and R₇ may include a hydrogen, alkyl, substituted alkyl, alkenyl, substituted alkenyl, alkynyl, substituted alkynyl, cycloalkyl, substituted cycloalkyl, cycloalkenyl and substituted cycloalkenyl; or R₆ and R₇ may be joined to form an alkylene or substituted alkylene group having from two to ten carbon atoms; R₈ may include an alkyl, substituted alkyl, alkenyl, substituted alkenyl, alkynyl, substituted alkynyl, cycloalkyl, substituted cycloalkyl, cycloalkenyl and substituted cycloalkenyl; and R₉ may include a hydrogen, alkyl, substituted alkyl, alkenyl, substituted alkenyl, alkynyl, substituted alkynyl, cycloalkyl, substituted cycloalkyl, cycloalkenyl and substituted cycloalkenyl; or R₈ and R₉ may be joined to form an alkylene or substituted alkylene group having from two to ten carbon atoms; or R₁ and R₂ may be selected from the group consisting of CH₃O—, C₅H₉O—, C₆H₅SO₂O—, CH₃CO—, C₆H₅SO₂NH—, (C₆H₅SO₂)₂N—, C₄H₈N—, C₅H₁₀N—, and C₅H₁₁NN—.

In another embodiment, a compound of Formula Ib is provided:

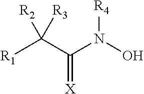

or a pharmaceutically acceptable salt thereof, wherein:

R₁ is selected from the group consisting of C₁₋₆alkyl, C₁₋₆substituted alkyl, C₂₋₆alkenyl, C₂₋₆substituted alkenyl, C₂₋₆alkynyl, C₂₋₆substituted alkynyl, C₃₋₆cycloalkyl, C₃₋₆substituted cycloalkyl, phenyl, cyano, hydroxyl, thiol, sulfonamide, amine, or:

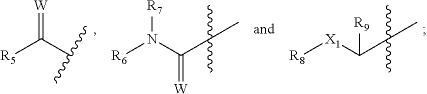

X is oxygen or sulfur;

X₁ is O, S, —S(O)— or —S(O)₂—;

W is oxygen or sulfur;

R₅ is selected from the group consisting of alkoxy, alkyl, substituted alkyl, alkenyl, substituted alkenyl, alkynyl, substituted alkynyl, cycloalkyl, substituted cycloalkyl, cycloalkenyl and substituted cycloalkenyl;

R₆ and R₇ are each independently selected from the group consisting of hydrogen, C₁₋₆alkyl, C₁₋₆substituted alkyl, C₂₋₆alkenyl, C₂₋₆substituted alkenyl, C₂₋₆alkynyl, C₂₋₆substituted alkynyl, C₃₋₆cycloalkyl, C₃₋₆substituted cycloalkyl; or R₆ and R₇ are joined to form an C₃₋₁₀cycloalkyl;

R₈ is selected from the group consisting of hydrogen, C₁₋₆alkyl, C₁₋₆substituted alkyl, C₂₋₆alkenyl, C₂₋₆substituted alkenyl, C₂₋₆alkynyl, C₂₋₆substituted alkynyl, C₃₋₆cycloalkyl, C₃₋₆ substituted cycloalkyl; and

R₉ is selected from the group consisting of hydrogen, C₁₋₆alkyl, C₁₋₆substituted alkyl, C₂₋₆alkenyl, C₂₋₆substituted alkenyl, C₂₋₆alkynyl, C₂₋₆substituted alkynyl, C₃₋₆cycloalkyl, C₃₋₆ substituted cycloalkyl;

R₂ is selected from the group consisting of hydrogen and C₁₋₆ alkyl;

R₃ is selected from the group consisting of C₁₋₆alkyl-NH—, NH₂—, -alkyl-C(O)—NH—, C₆H₅SO₂NH—, (C₆H₅SO₂)₂N—, C₄H₈N—, and C₅H₁₁NN—;

R₄ is selected from the group consisting of hydrogen, C₁₋₆alkyl, C₁₋₆substituted alkyl.

In some embodiments, R₂ may be hydrogen. In another embodiment, R₄ may be H, or R₄ may be a lower alkyl group, e.g., methyl, ethyl, propyl, isobutyl, t-butyl, n-butyl, isopropyl, etc.

In certain embodiments, X is oxygen. In another embodiment, R₃ may be NH₂ or CH₃—C(O)—NH—.

R₁ may be an alkyl group, e.g. a straight or branched alkyl, such as iso-butyl, propyl, ethyl, methyl, t-butyl, n-butyl, etc. In some embodiments, R₂ and R₃ are connected to a chiral center.

In another of its composition aspects, the present disclosure is directed to pharmaceutical compositions comprising a pharmaceutically acceptable carrier and a pharmaceutically effective amount of a compound of Formula Ia or Ib.

Accordingly, in one of its method aspects, the present disclosure is directed to a method for treating a mammal with a degenerative disease. The method includes administering to the mammal a pharmaceutical composition comprising a pharmaceutically acceptable carrier and an effective cytoprotective amount of a compound of Formula Ia or Ib above.

For purposes of this description hydroxamic acid compounds of Formula I are named using conventional amino hydroxamic acid nomenclature, i.e., the nitrogen atom of the hydroxylamine bonded to the carbonyl group (C═O) is designated in the Formula Ia or Ib.

In some cases, the 3,4,5,-trisubstituted aryl amino hydroxamic acid of the present disclosure may include one or more chiral centers. Such compounds may be prepared as a racemic mixture. If desired, however, such compounds may be prepared or isolated as pure stereoisomers, i.e., as individual enantiomers or diastereomers, or as stereoisomer-enriched mixtures. All such stereoisomers and enriched mixtures of the alkyl amino hydroxamic acid of Formula Ia and Ib are included within the scope of the present disclosure. Pure stereoisomers or enriched mixtures may be prepared using, for example, optically active starting materials or stereoselective reagents well known in the art. Alternatively, racemic mixtures of such compounds may be separated using, for example, chiral column chromatography, chiral resolving agents and the like.

When describing the alkyl amino hydroxamic acid, pharmaceutical compositions and methods of this invention, the following terms have the following meanings unless otherwise specified.

“Acyl” refers to the group —C(O)R where R is hydrogen, alkyl, aryl or cycloalkyl.

“Acylamino” refers to the group —NRC(O)R where each R is independently hydrogen, alkyl, aryl or cycloalkyl.

“Acyloxy” refers to the group —OC(O)R where R is hydrogen, alkyl, aryl or cycloalkyl.

“Alkenyl” refers to a monovalent branched or unbranched unsaturated hydrocarbon group preferably having from 2 to 10 carbon atoms and more preferably 2 to 6 carbon atoms and having at least 1 and preferably from 1 to 2 sites of carbon-carbon double bond unsaturation. Preferred alkenyl groups include ethenyl (—CH═CH2), n-propenyl (—CH2CH═CH2), isopropenyl (—C(CH3)=CH2), and the like.

“Substituted alkenyl” refers to an alkenyl group having from 1 to 5 substituents, and preferably from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Alkoxy” refers to the group —OR where R is alkyl. Preferred alkoxy groups include, by way of example, methoxy, ethoxy, n-propoxy, isopropoxy, n-butoxy, tert-butoxy, sec-butoxy, n-pentoxy, n-hexoxy, 1,2-dimethylbutoxy, and the like.

“Substituted alkoxy” refers to an alkoxy group having from 1 to 5 substituents, and preferably from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Alkoxycarbonyl” refers to the group —C(O)OR where R is alkyl or cycloalkyl.

“Alkoxycarbonylamino” refers to the group —NRC(O)OR′ where R is hydrogen, alkyl, aryl or cycloalkyl, and R′ is alkyl or cycloalkyl.

“Alkyl” refers to a monovalent branched or unbranched saturated hydrocarbon group preferably having from 1 to about 10 carbon atoms, more preferably from 1 to 8 carbon atoms and still more preferably 1 to 6 carbon atoms. This term is exemplified by groups such as methyl, ethyl, n-propyl, isopropyl, n-butyl, isobutyl, tert-butyl, n-hexyl, n-octyl, tert-octyl and the like. The term “lower alkyl” refers to an alkyl group having from 1 to 6 carbon atoms.

“Substituted alkyl” refers to an alkyl group having from 1 to 5 substituents, and preferably from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Alkylene” refers to a divalent branched or unbranched saturated hydrocarbon group preferably having from 1 to 10 carbon atoms and more preferably from 1 to 6 carbon atoms. This term is exemplified by groups such as methylene (—CH₂—), ethylene (—CH₂CH₂—), the propylene isomers (e.g., —CH₂CH₂CH₂— and —CH(CH₃)CH₂—) and the like.

“Substituted alkylene” refers to an alkylene group having from 1 to 5 substituents, and preferably from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂-“Alkynyl” refers to a monovalent branched or unbranched unsaturated hydrocarbon group preferably having from 2 to 10 carbon atoms and more preferably 2 to 6 carbon atoms and having at least 1 and preferably from 1-2 sites of carbon-carbon triple bond unsaturation. Preferred alkynyl groups include ethynyl (—C═CH), propargyl (—CH₂C═CH) and the like.

“Substituted alkynyl” refers to an alkynyl group having from 1 to 5 substituents, and preferably from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Amino” refers to the group —NH₂.

“Substituted amino” refers to the group —N(R)₂ where each R is independently selected from the group consisting of hydrogen, alkyl, substituted alkyl, alkenyl, substituted alkenyl, alkynyl, substituted alkynyl, aryl, cycloalkyl, substituted cycloalkyl, and where both R groups are joined to form an alkylene group. When both R groups are hydrogen, —N(R)₂ is an amino group.

“Aminocarbonyl” refers to the group —C(O)NRR where each R is independently hydrogen, alkyl, aryl and cycloalkyl, or where the R groups are joined to form an alkylene group.

“Aminocarbonylamino” refers to the group —NRC(O)NRR where each R is independently hydrogen, alkyl, aryl or cycloalkyl, or where two R groups are joined to form an alkylene group.

“Aminocarbonyloxy” refers to the group —OC(O)NRR where each R is independently hydrogen, alkyl, aryl or cycloalkyl, or where the R groups are joined to form an alkylene group.

“Aryl” refers to an unsaturated aromatic carbocyclic group of from 6 to 14 carbon atoms having a single ring (e.g., phenyl) or multiple condensed rings (e.g., naphthyl or anthryl). Preferred aryls include phenyl, naphthyl and the like. Unless otherwise constrained by the definition for the individual substituent, such aryl groups can optionally be substituted with from 1 to 5 substituents, preferably 1 to 3 substituents, selected from the group consisting of acyl, acylamino, acyloxy, alkenyl, substituted alkenyl, alkoxy, substituted alkoxy, alkoxycarbonyl, alkyl, substituted alkyl, alkynyl, substituted alkynyl, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, nitro, thioalkoxy, substituted thioalkoxy, thioaryloxy, thiol, alkyl-S(O)—, aryl-S(O)—, alkyl-S(O)₂— and aryl-S(O)₂—.

“Aryloxy” refers to the group —OR where R is aryl.

“Cycloalkyl” refers to a cyclic alkyl group of from 3 to 10 carbon atoms having a single cyclic ring or multiple condensed or bridged rings which can be optionally substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, or multiple or bridged ring structures such as adamantanyl and the like. The term “lower cycloalkyl” refers to a cycloalkyl group having from 3 to 6 carbon atoms.

“Substituted cycloalkyl” refers to a cycloalkyl group having from 1 to 5 substituents, and preferably from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Cycloalkoxy” refers to the group —OR where R is cycloalkyl. Such cycloalkoxy groups include, by way of example, cyclopentoxy, cyclohexoxy and the like.

“Cycloalkenyl” refers to a cyclic alkenyl group of from 4 to 10 carbon atoms having a single cyclic ring and at least one point of internal unsaturation which can be optionally substituted with from 1 to 3 alkyl groups. Examples of suitable cycloalkenyl groups include, for instance, cyclopent-3-enyl, cyclohex-2-enyl, cyclooct-3-enyl and the like.

“Substituted cycloalkenyl” refers to a cycloalkenyl group having from 1 to 5 substituents, and preferably from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Halo” or “halogen” refers to fluoro, chloro, bromo and iodo.

“Hydroxyl” refers to the group, —OH.

“Pharmaceutically-acceptable salt” refers to any salt of a compound of this invention which retains its biological properties and which is not biologically or otherwise undesirable. Such salts may be derived from a variety of organic and inorganic counter-ions well known in the art and include, by way of example, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like. The term “pharmaceutically-acceptable cation” refers to a pharmaceutically acceptable cationic counter-ion of an acidic functional group. Such cations may include sodium, potassium, calcium, magnesium, ammonium, tetraalkylammonium cations, and the like.

The amino alkyl/aryl hydroxamic acids disclosed herein may be prepared from readily available starting materials using the following general methods and procedures. It should be appreciated that, where typical or preferred process conditions (i.e., reaction temperatures, times, mole ratios of reactants, solvents, pressures, etc.) are described, other process conditions may also be used unless otherwise stated. Optimum reaction conditions may vary with the particular reactants or solvent used, but such conditions may be determined by one skilled in the art by routine optimization procedures.

Additionally, as will be apparent to those skilled in the art, conventional protecting groups may be used to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and G. M. Wuts, Protecting Groups in Organic Synthesis, Second Edition, Wiley, New York, 1991, and references cited therein.

In an embodiment, a method of synthesis of an amino alkyl/aryl hydroxamic acid includes coupling a (Boc) protected amino alkyl/aryl carbonyl compound of Formula II with hydroxylamine of Formula III:

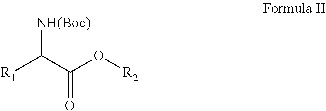

where R1 is as described above and R2 is lower alkyl such as a methyl or ethyl group. Formula III can be represented by:

HO—NH—R3

where R3 is an amino group, and R1 or R2 may be a lower alkyl such as methyl, ethyl propyl, or halogen group such as fluoro, chloro and bromo, under conventional reaction conditions.

This coupling reaction may be conducted by contacting the aryl carbonyl compound of Formula II with at least one equivalent, and, in an embodiment, about 12 to about 15 equivalents, of hydroxylamine of Formula III in an inert polar solvent such as methanol, ethanol, 1,4-dioxane, tetrahydrofuran, dimethyl sulfoxide, dimethylformamide and the like. This reaction may be conducted at a temperature of from about 0° C. to about 45° C. for about 1 to about 4 hours. Optionally, a catalytic amount of an acid, such as hydrochloric acid, acetic acid, silica gel and the like, may be employed in this reaction. Upon completion of the reaction, the amino alkylaryl hydroxamic acid of Formula I is recovered by conventional methods including precipitation, chromatographic separation, filtration, distillation, sublimation, and the like.

The amino alkyl/aryl carbonyl compounds of formula I employed in the above-described coupling reaction may include known compounds or compounds that can be prepared from known compounds by conventional procedures. For example, amino alkyl/aryl carbonyl compounds of formula I where R1 is —CO(O)R2 are readily prepared by acylation of the corresponding Boc-amino carboxylic acid. For example, in an embodiment, L-Boc leucine methyl ester (available from Aldrich Chemical Co., 1001 W. St. Paul Avenue, Milwaukee, Wis., USA 53233-2641 and CS Bio, Menlo Park, Calif.) is acetylated by e.g., contacting the ester with excess acetic anhydride in the presence of an acid catalyst, such as perchloric acid, followed by N-acylation of the intermediate hydroxamic acid, to obtain Boc-protected amino alkyl/aryl hydroxamic acid. The Boc group may be removed in the presence of trifluoroacetic acid in methanol.

The hydroxylamine compounds of Formula III are also known compounds or compounds which can be prepared from known compounds by conventional procedures. Typically, the hydroxylamine compounds of Formula III may be prepared by reducing the corresponding nitro compound (i.e., R4-NO2, where R4 is as defined above) using a suitable reducing agent such as activated zinc/acetic acid, activated zinc/ammonium chloride or an aluminum/mercury amalgam. This reaction is typically conducted at a temperature ranging from about 15° C. to about 100° C. for about 0.5 to 12 hours, or, in an embodiment, about 2 to about 6 hours, in an aqueous reaction media, such as an alcohol/water mixture in the case of the zinc reagents or an ether/water mixture in the case of the aluminum amalgams. Aliphatic nitro compounds (in the form of their salts) may also be reduced to hydroxylamines using borane in tetrahydrofuran. Since some hydroxylamines have limited stability, such compounds may generally be prepared immediately prior to reaction with the aryl carbonyl compound of Formula V.

Accordingly, compositions of the present disclosure may include the following compounds as set forth in Table I below: AK-10—2-amino-N-hydroxy-4-methylpentamide (Salt TFA); AK-12—2-acetoamido-N-hydroxy-4-methylpentamide; AK-25—2-amino-N-hydroxypentamide (Salt TFA); AK-26—3-amino-N-hydroxy-4-methylpentamide (Salt TFA); AK-27—2-amino-N-hydroxypropanamide (Salt TFA); AK-28—2-amino-N-hydroxybutanamide (Salt TFA); AK-29—2-amino-N-hydroxy-3-methylpentamide (Salt TFA); AK-30—2-amino-N-hydroxy-4-methylpentamide (Salt TFA), and pharmaceutically acceptable salts thereof. Table I sets forth the structures of the compounds and the degree to which these compounds are neuroprotective under 24-hour incubation with 400 μM 3-aminopropanal in retinal ganglion cells.

TABLE I

| | | % Neuroprotection |
|---|---|---|
| Compound | Structure | (600 μM) |
| AK-10 (TFA salt) | 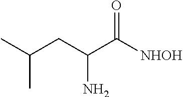 | 100 |
| AK-12 | 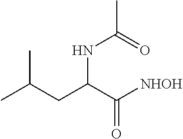 | 10 |
| AK-25 (TFA salt) | 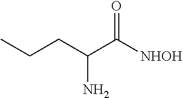 | 28 |
| AK-26 (TFA salt) | 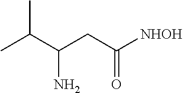 | 58 |
| AK-27 (TFA salt) | 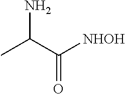 | 6 |
| AK-28 (TFA salt) | 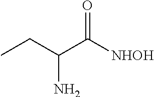 | 39 |
| AK-29 (TFA salt) | 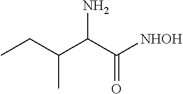 | 4 |
| AK-30 (TFA salt) | 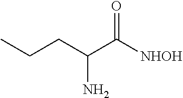 | 2 |

### Pharmaceutical Compositions

When employed as pharmaceuticals, the hydroxamic acids described herein may be administered in the form of a pharmaceutical composition. Such compositions comprise at least one active compound and may be prepared using procedures well known in the pharmaceutical art.

Generally, the compounds of the present disclosure are administered in a pharmaceutically effective amount. The amount of the compound actually administered will typically be determined by a physician, based on the relevant circumstances, including the condition to be treated, the chosen route of administration, the actual compound administered, the age, weight, and response of the individual patient, the severity of the patient's symptoms, and the like.

The pharmaceutical compositions of the present disclosure may be administered by any suitable route including, by way of illustration, oral, topical, rectal, transdermal, subcutaneous, intravenous, intramuscular, intranasal, and the like. Depending on the intended route of delivery, in an embodiment, the compounds of this invention the present disclosure may be formulated as either oral, topical or injectable compositions.

Any suitable sustained release materials may be used in the compositions of the present disclosure.

The compounds described herein are suitable for use in a variety of drug delivery systems. The compounds of the present disclosure may be formulated in any suitable pharmaceutical composition including tablets, capsules, liquid, injection and ointment.

Pharmaceutical compositions for oral administration may be formulated as bulk liquid solutions or suspensions, or bulk powders. Such compositions may be administered in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms may include pre-filled, pre-measured ampules or syringes of the liquid compositions or pills, tablets, capsules or the like in the case of solid compositions.

Liquid forms suitable for oral administration may include any suitable aqueous or nonaqueous vehicle with buffers, suspending and dispensing agents, colorants, flavors and the like. Solid forms may include, for example, any of the following ingredients, or similar compounds of a similar nature: any suitable binder such as microcrystalline cellulose, gum tragacanth or gelatin; any suitable excipient such as starch or lactose; any suitable disintegrating agent such as alginic acid, Primogel®, or corn starch; any suitable lubricant such as magnesium stearate; any suitable glidant such as colloidal silicon dioxide; any suitable sweetening agent such as sucrose or saccharin; or any suitable flavoring agent such as peppermint, methyl salicylate, or orange flavoring.

Topical compositions are typically formulated as a topical ointment or cream containing the active ingredient(s), generally in an amount ranging from about 0.01% to about 20% by weight, in an embodiment from about 0.1% to about 10% by weight, and in anotheran embodiment, from about 0.5% to about 15% by weight. The active ingredients may be formulated as an ointment, in which the active ingredients may be combined with either a paraffinic, a water-miscible or any other suitable ointment base. The active ingredients may be formulated in a cream with, for example, an oil-in-water cream base or any other suitable cream base. Such topical formulations are well-known in the art and generally include additional ingredients to enhance the dermal penetration or stability of the active ingredients or the formulation. All such known topical formulations and ingredients may be included within the scope of this disclosure.

The compounds of the present disclosure may be administered using any suitable delivery mechanism. Accordingly, topical administration may be accomplished by a transdermal device such as a patch in any suitable form such as a reservoir or porous membrane or of a solid matrix type.

Injectable compositions may include injectable sterile saline or phosphate-buffered saline or other suitable injectable carriers known in the art. As before, the alkyl nitrone compound be included in such compositions maybe, in an embodiment, from about 0.05% to about 10% by weight with the remainder being the injectable carrier and the like.

The above-described components for orally and topically administrable or injectable compositions are merely representative. Other materials as well as processing techniques and the like as set forth in Part 8 of Remington's Pharmaceutical Sciences, 18th edition, 1990, Mack Publishing Company, Easton, Pa., 18042, which is incorporated herein by reference may be employed in the disclosed compositions.

The compounds of the present disclosure may also be administered in sustained release forms or from sustained release drug delivery systems. Representative sustained release materials can be found in Remington's Pharmaceutical Sciences incorporated herein.

The compound of Formula I may, in an embodiment, be dissolved in a buffered sterile saline injectable aqueous medium to an appropriate concentration.

The following synthetic and biological examples are offered to illustrate the present disclosure and are not to be construed in any way as limiting the scope of the present disclosure.

### EXAMPLES

It should be understood, that the following abbreviations have the following meanings in the examples below. Abbreviations not defined below have their generally accepted meaning. All temperatures in the examples below are in degrees Celsius (° C.) (unless otherwise indicated).

Examples I-VIII describe the synthesis of intermediates useful for preparing hydroxamic acids disclosed herein and the synthesis of various hydroxamic acids. Examples of suitable hydroxylamines for the uses described herein include, but are not limited to, N-isopropylhydroxylamine, N-n-propylhydroxylamine, N-n-butylhydroxylamine, N-tert-butylhydroxylamine, N-cyclohexylhydroxylamine and the like. Examples IX-XI describe the testing of such compounds.

### Example I

### 2-amino-N-hydroxy-4-methylpentamide (TFA salt) (AK-10)

Hydroxylamine hydrochloride (5.55 g, 80.0 mmol) in methanol (20 ml) was mixed with KOH (5.06 g, 90.0 mmol) at 40° C. in methanol (60 ml), cooled to 0° C., and filtered. The tert-butyl 1-(methoxycarbonyl)-3-methylbutylcarbamate (0.735 g, 3.0 mmol) was then added to the filtrate followed by addition (over 20 min) of KOH (0.050 g, 0.001 mmol). The mixture was stirred at room temperature for 1 h. The mixture was added to stirring cold water (100 ml), and the pH was adjusted to 7 by adding acetic acid. The precipitate was filtered off, and the resulting product was dried in a vacuum oven at 40° C. overnight to yield tert-butyl 1-(hydroxycarbamoyl)-3-methylbutylcarbamate (90%). The resulting compound had a melting point of about 104-105° C.; and an NMR spectrum of 1H NMR (400 MHz, DMSO-d6) δ 8.90 (s, 10H), 6.90 (s, 1 NH), 3.90 (s, 1H), 3.30 (s, 1H), 1.83 (m, 1H), 1.56 (d, 2H), 1.40 (s, 9H), 0.90 (d, 6H).

To a solution of tert-butyl 1-(hydroxycarbamoyl)-3-methylbutylcarbamate compounds (1.0 eq) in anhydrous dichloromethane (3.0 ml) was added a solution of TFA (5.0 eq). The resulting mixture was stirred under nitrogen at room temperature and monitored by TLC. After completion of the reaction, the resulting mixture was concentrated in vacuo and crystallized in methanol: ether (3 ml:15 ml). The resulting product was washed with ether (2×20 ml) and then dried in desiccators to yield AK-10 (90%). The resulting compound had a melting point of about 145-147° C.; and an NMR spectrum of 1H NMR (400 MHz, DMSO-d6) δ 10.32 (s, 1H), 9.38 (s, 1 OH), 8.40 (s, 1 NH), 3.73 (s, 1H), 3.37 (s, 2H), 1.83 (m, 1H), 1.56 (d, 2H), 0.95 (d, 6H).

### Example II

### 2-acetoamido-N-hydroxy-4-methylpentamide (AK-12)

Hydroxylamine hydrochloride (5.55 g, 80.0 mmol) in methanol (20 ml) was mixed with KOH (5.06 g, 90.0 mmol) at 40° C. in methanol (60 ml), cooled to 0° C., and filtered. The methyl 2-acetoamido-4-methylpentanoate (0.561 g, 3.0 mmol) was then added to the filtrate followed by addition (over 20 min) of KOH (0.050 g, 0.001 mmol). The mixture was stirred at room temperature for 1 h. The mixture was added to stirring cold water (100 ml), and the pH was adjusted to 7 by adding acetic acid. The precipitate was filtered off, and the resulting product was dried in a vacuum oven at 40° C. overnight to yield 2-aceto-N-hydroxy-4-methylpentamide AK-12 (90%). The resulting compound had a melting point of about 47-49° C. and an NMR spectrum of 1H NMR (400 MHz, DMSO-d6) δ 8.90 (s, 10H), 6.90 (s, 1 NH), 3.90 (s, 1H), 3.30 (s, 1H), 1.83 (m, 1H), 1.56 (d, 2H), 1.40 (s, 9H), 0.90 (d, 6H).

### Example III

### 2-amino-N-hydroxypentamide (TFA salt) (AK-25)

Hydroxylamine hydrochloride (5.55 g, 80.0 mmol) in methanol (20 ml) was mixed with KOH (5.06 g, 90.0 mmol) at 40° C. in methanol (60 ml), cooled to 0° C., and filtered. The tert-butyl 1-(methoxycarbonyl)-2-methylpropylcarbamate (0.693 g, 3.0 mmol) was then added to the filtrate followed by addition (over 20 min) of KOH (0.050 g, 0.001 mmol). The mixture was stirred at room temperature for 1 h. The mixture was added to stirring cold water (100 ml), and the pH was adjusted to 7 by adding acetic acid. The precipitate was filtered off, and the resulting product was dried in a vacuum oven at 40° C. overnight to yield tert-butyl 1-(hydroxycarbamoyl)-2-methylprop

The present disclosure relates to the prevention of and treatment of degenerative diseases characterized by the reduction of a specific cell population by the excessive production of reactive aldehydes.ylcarbamate (90%). The resulting compound had an NMR spectrum of 1H NMR (400 MHz, DMSO-d6) δ 8.90 (s, 10H), 6.90 (s, 1 NH), 3.90 (s, 1H), 3.30 (s, 1H), 1.83 (m, 1H), 1.56 (d, 2H), 1.40 (s, 9H), 0.90 (d, 6H).

To a solution of tert-butyl 1-(hydroxycarbamoyl)-2-methylpropylcarbamate compound (1.0 eq) in anhydrous dichloromethane (3.0 ml) was added a solution of TFA (5.0 eq). The resulting mixture was stirred under nitrogen at room temperature and monitored by TLC. After completion of reaction it was concentrated in vacuum and crystallized in methanol: ether (3 ml:15 ml). The resulted product was washed with ether (2 X×20 ml) and then dried in desiccators to yield AK-25 (90%). The resulting compound had a melting point of about 152-154° C., and an NMR spectrum of 1H NMR (400 MHz, DMSO-d6) δ 10.32 (s, 1H), 9.38 (s, 10H), 8.40 (s, 1 NH), 3.73 (s, 1H), 3.37 (s, 2H), 1.83 (m, 1H), 1.56 (d, 2H), 0.95 (d, 6H).

### Example IV

### 3-amino-N-hydroxy-4-methylpentamide (TFA salt) (AK-26)

Hydroxylamine hydrochloride (5.55 g, 80.0 mmol) in methanol (20 ml) was mixed with KOH (5.06 g, 90.0 mmol) at 40° C. in methanol (60 ml), cooled to 0° C., and filtered. The tert-butyl 1-(methoxycarbonyl)-3-methylbutanyl-2-carbamate (0.735 g, 3.0 mmol) was then added to the filtrate followed by addition (over 20 min) of KOH (0.050 g, 0.001 mmol). The mixture was stirred at room temperature for 1 h. The mixture was added to stirring cold water (100 ml), and the pH was adjusted to 7 by adding acetic acid. The precipitate was filtered off, and the resulting product was dried in a vacuum oven at 40° C. overnight to yield tert-butyl 1-(hydroxycarbamoyl)-3-methylbutanyl-2-carbamate (90%). The resulting compound had an NMR spectrum of 1H NMR (400 MHz, DMSO-d6) δ 8.90 (s, 10H), 6.90 (s, 1 NH), 3.90 (s, 1H), 3.30 (s, 1H), 1.83 (m, 1H), 1.56 (d, 2H), 1.40 (s, 9H), 0.90 (d, 6H).

To a solution of tert-butyl 1-(hydroxycarbamoyl)-3-methylbutanyl-2-carbamate compound (1.0 eq) in anhydrous dichloromethane (3.0 ml) was added a solution of TFA (5.0 eq). The resulting mixture was stirred under nitrogen at room temperature and monitored by TLC. After completion of reaction it was concentrated in vacuo and crystallized in methanol: ether (3 ml:15 ml). The resulted product was washed with ether (2×20 ml) and then dried in desiccators to yield AK-26 (90%). The resulting compound had an NMR spectrum of 1H NMR (400 MHz, DMSO-d6) δ 10.32 (s, 1H), 9.38 (s, 10H), 8.40 (s, 1 NH), 3.73 (s, 1H), 3.37 (s, 2H), 1.83 (m, 1 H), 1.56 (d, 2H), 0.95 (d, 6H).

### Example V

### 2-amino-N-hydroxypropanamide (TFA salt) (AK-27)

Hydroxylamine hydrochloride (5.55 g, 80.0 mmol) in methanol (20 ml) was mixed with KOH (5.06 g, 90.0 mmol) at 40° C. in methanol (60 ml), cooled to 0° C., and filtered. The tert-butyl 1-(methoxycarbonyl)ethylcarbamate (0.609 g, 3.0 mmol) was then added to the filtrate followed by addition (over 20 min) of KOH (0.050 g, 0.001 mmol). The mixture was stirred at room temperature for 1 h. The mixture was added to stirring cold water (100 ml), and the pH was adjusted to 7 by adding acetic acid. The precipitate was filtered off, and the resulting product was dried in a vacuum oven at 40° C. overnight to yield tert-butyl 1-(hydroxycarbamoyl)ethylcarbamate (90%). The resulting compound had an NMR spectrum of 1H NMR (400 MHz, DMSO-d6) δ 8.90 (s, 10H), 6.90 (s, 1 NH), 3.90 (s, 1H), 3.30 (s, 1H), 1.83 (m, 1H), 1.56 (d, 2H), 1.40 (s, 9H), 0.90 (d, 6H).

To a solution of tert-butyl 1-(hydroxycarbamoyl)ethylcarbamate compound (1.0 eq) in anhydrous dichloromethane (3.0 ml) was added a solution of TFA (5.0 eq). The resulting mixture was stirred under nitrogen at room temperature and monitored by TLC. After completion of reaction it was concentrated in vacuo and crystallized in methanol: ether (3 ml:15 ml). The resulting product was washed with ether (2×20 ml) and then dried in desiccators to yield AK-27 (90%). The resulting compound had a melting point of about 74-76° C. and an NMR spectrum of 1H NMR (400 MHz, DMSO-d6) δ 10.32 (s, 1H), 9.38 (s, 10H), 8.40 (s, 1 NH), 3.73 (s, 1H), 3.37 (s, 2H), 1.83 (m, 1H), 1.56 (d, 2H), 0.95 (d, 6H).

### Example VI

### 2-amino-N-hydroxybutanamide (TFA salt) (AK-28)

Hydroxylamine hydrochloride (5.55 g, 80.0 mmol) in methanol (20 ml) was mixed with KOH (5.06 g, 90.0 mmol) at 40° C. in methanol (60 ml), cooled to 0° C., and filtered. The tert-butyl 1-(methoxycarbonyl)propylcarbamate (0.651 g, 3.0 mmol) was then added to the filtrate followed by addition (over 20 min) of KOH (0.050 g, 0.001 mmol). The mixture was stirred at room temperature for 1 h. The mixture was added to stirring cold water (100 ml), and the pH was adjusted to 7 by adding acetic acid. The precipitate was filtered off, and the resulting product was dried in a vacuum oven at 40° C. overnight to yield tert-butyl 1-(hydroxycarbamoyl)propylcarbamate (90%). The resulting compound had an NMR spectrum of 1H NMR (400 MHz, DMSO-d6) δ 8.90 (s, 10H), 6.90 (s, 1 NH), 3.90 (s, 1H), 3.30 (s, 1H), 1.83 (m, 1H), 1.56 (d, 2H), 1.40 (s, 9H), 0.90 (d, 6H).

To a solution of tert-butyl 1-(hydroxycarbamoyl)propylcarbamate compound (1.0 eq) in anhydrous dichloromethane (3.0 ml) was added a solution of TFA (5.0 eq). The resulting mixture was stirred under nitrogen at room temperature and monitored by TLC. After completion of reaction it was concentrated in vacuo and crystallized in methanol: ether (3 ml:15 ml). The resulted product was washed with ether (2×20 ml) and then dried in desiccators to yield AK-28 (90%). The resulting compound had an NMR spectrum of 1H NMR (400 MHz, DMSO-d6) δ 10.32 (s, 1H), 9.38 (s, 10H), 8.40 (s, 1 NH), 3.73 (s, 1H), 3.37 (s, 2H), 1.83 (m, 1H), 1.56 (d, 2H), 0.95 (d, 6H).

### Example VII

### 2-amino-N-hydroxy-3-methylpentamide (TFA salt) (AK-29)

Hydroxylamine hydrochloride (5.55 g, 80.0 mmol) in methanol (20 ml) was mixed with KOH (5.06 g, 90.0 mmol) at 40° C. in methanol (60 ml), cooled to 0° C., and filtered. The tert-butyl 1-(methoxycarbonyl)-2-methylbutylcarbamate (0.735 g, 3.0 mmol) was then added to the filtrate followed by addition (over 20 min) of KOH (0.050 g, 0.001 mmol). The mixture was stirred at room temperature for 1 h. The mixture was added to stirring cold water (100 ml), and the pH was adjusted to 7 by adding acetic acid. The precipitate was filtered off, and the resulting product was dried in a vacuum oven at 40° C. overnight to yield tert-butyl 1-(hydroxycarbamoyl)-2-methylbutylcarbamate (90%). The resulting compound had an NMR spectrum of 1H NMR (400 MHz, DMSO-d6) δ 8.90 (s, 10H), 6.90 (s, 1 NH), 3.90 (s, 1H), 3.30 (s, 1H), 1.83 (m, 1H), 1.56 (d, 2H), 1.40 (s, 9H), 0.90 (d, 6H).

To a solution of tert-butyl 1-(hydroxycarbamoyl)-2-methylbutylcarbamate compound (1.0 eq) in anhydrous dichloromethane (3.0 ml) was added a solution of TFA (5.0 eq). The resulting mixture was stirred under nitrogen at room temperature and monitored by TLC. After completion of reaction it was concentrated in vacuo and crystallized in methanol: ether (3 ml:15 ml). The resulted product was washed with ether (2×20 ml) and then dried in desiccators to yield AK-29 (90%). The resulting compound had an NMR spectrum of 1H NMR (400 MHz, DMSO-d6) δ 10.32 (s, 1H), 9.38 (s, 10H), 8.40 (s, 1 NH), 3.73 (s, 1H), 3.37 (s, 2H), 1.83 (m, 1 H), 1.56 (d, 2H), 0.95 (d, 6H).

### Example VIII

### 2-amino-N-hydroxy-4-methylpentamide (TFA salt) (AK-30) [difference between AK-10 vs AK-30]

Hydroxylamine hydrochloride (5.55 g, 80.0 mmol) in methanol (20 ml) was mixed with KOH (5.06 g, 90.0 mmol) at 40° C. in methanol (60 ml), cooled to 0° C., and filtered. The tert-butyl 1-(methoxycarbonyl)pentylcarbamate (0.735 g, 3.0 mmol) was then added to the filtrate followed by addition (over 20 min) of KOH (0.050 g, 0.001 mmol). The mixture was stirred at room temperature for 1 h. The mixture was added to stirring cold water (100 ml), and the pH was adjusted to 7 by adding acetic acid. The precipitate was filtered off, and the resulting product was dried in a vacuum oven at 40° C. overnight to yield tert-butyl 1-(hydroxycarbamoyl)pentylcarbamate (90%). The resulting compound had an NMR spectrum of 1H NMR (400 MHz, DMSO-d6) δ 8.90 (s, 10H), 6.90 (s, 1 NH), 3.90 (s, 1H), 3.30 (s, 1H), 1.83 (m, 1H), 1.56 (d, 2H), 1.40 (s, 9H), 0.90 (d, 6H).

To a solution of tert-butyl 1-(hydroxycarbamoyl)pentylcarbamate compound (1.0 eq) in anhydrous dichloromethane (3.0 ml) was added a solution of TFA (5.0 eq). The resulting mixture was stirred under nitrogen at room temperature and monitored by TLC. After completion of reaction it was concentrated in vacuo and crystallized in methanol: ether (3 ml:15 ml). The resulting product was washed with ether (2×20 ml) and then dried in desiccators to yield AK-30 (90%). The resulting compound had an NMR spectrum of 1H NMR (400 MHz, DMSO-d6) δ 10.32 (s, 1H), 9.38 (s, 10H), 8.40 (s, 1 NH), 3.73 (s, 1H), 3.37 (s, 2H), 1.83 (m, 1H), 1.56 (d, 2H), 0.95 (d, 6H).

### Example IX

### Biology: Retinal Cell Cultures

The rat retinal cell line, E1A-NR.3, was grown in DMEM, containing 10% FBS, in 75 cm2 flasks. For neurotoxicity assays, cells were plated in 48-well tissue culture plates and exposed to 3-aminopropanal in DMEM for 24 hours. Media was collected and assayed for LDH using the Roche assay kit. Rabbit muscle LDH was used for the standard curve. Drug treatments of aldehyde sequestering agents were as co-treatments or delayed administration of about 0.5 to about 3 hours. All drugs were dissolved in PBS.

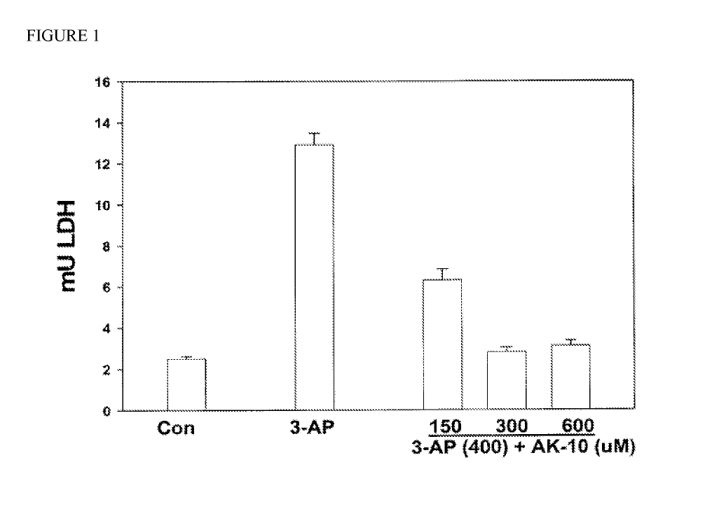

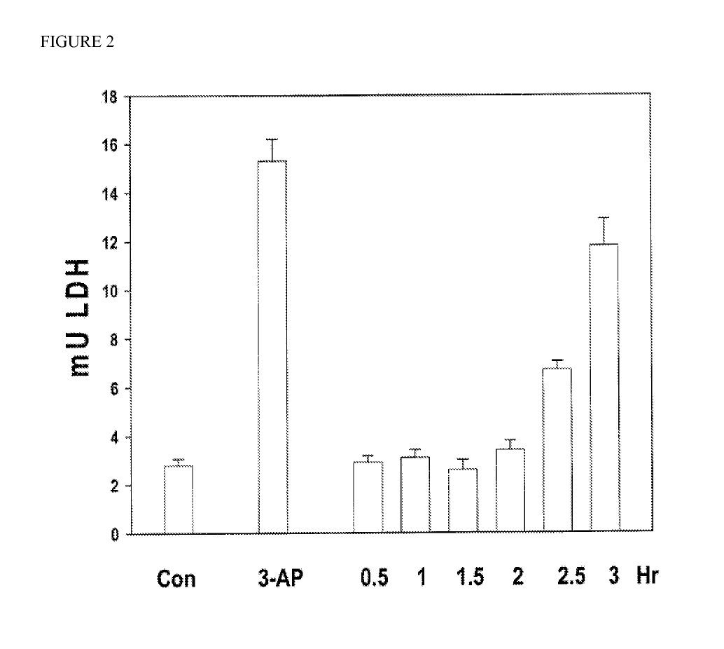

The hydroxylamines N-benzylhydroxylamine, cyclohexylhydroxylamine, t-butylhydroxylamine all protected retinal cells from 3-aminopropanal toxicity in the retinal cell line E1A-NR.3 as co-treatments and with delayed addition up to about 3 hours post 3-aminopropanal (1). The sulfhydral agent N-(2-mercaptopropionyl)-glycine is also active in this model of aldehyde-induced cell death while antioxidants, free radical scavengers and anti-inflammatory agents are inactive (1). AK-10 and analogs demonstrated activity in this model also, with AK-10 representing the most efficacious analog (Table 1; FIG. 1; FIG. 2).

As illustrated in FIG. 1, AK-10 was effective in reducing LDH levels in the EIA-NR.3 rat retinal cell time. AK-10 was combined with 400 μm 3-aminoproponal (3-AP) of concentrations of 150 μM, 300 μM and 600 μM AK-10 to significantly reduce the 24-hour level in cells exposed to SAP alone at all three doses.

FIG. 2 illustrates the duration of the effect of treatment with 300 μM AK-10 in EIA-NR.3 retinal cells treated with 3-AP. 24-hour LDH levels were increased after 0.5 hours, 1.0 hours, 1.5, 2, 2.5 and 3 hours demonstrating reduced LDH levels for up to at least 3 hours.

### Example X

### Trimethyltin (TMT) Model

Male Sprague Dawley rats (200 g; Harlan) were administered 8 mg, sc trimethyltin (TMT) and housed individually as a result of the aggressive behavior induced by the neurotoxicant. The day following the TMT treatment, rats were started on a once daily dosing for 17 days with vehicle or AK-10 (25 mg/kg, sc) in PBS. Rats were decapitated on day 19 and the hippocampi isolated and placed in chilled Hanks Balanced Salt Solution containing 20 mM HEPES (HBSS-HEPES, 4° C.) for release experiments.

Chilled tissues were cut into 300 μm slices. A single slice was incubated in 2 ml of HBSS-HEPES in 12-well culture plates at 37° C. for 30 min. The media was discarded and the slice incubated for 2-10 min periods, each with fresh media. Next, the slices were incubated for 5 min after which the media was isolated for amino acid analyses (Pre-sample), followed by a 5 min incubation in which 50 mM KCl was added to evoke neurotransmitter release from the slices (KCl sample). Next, a final 5 mM incubation in HBSS-HEPES was collected (Post sample).

The amino acids released into the media were isolated by cation exchange chromatography. Briefly, samples were transferred to 18×100 mm glass tubes containing the stable isotope internal standards 2.5 nmol [2H6]GABA and 5 nmol [2H5]glutamate and then 0.5 ml of the cation-exchange resin, Dowex AG 50W-X8, added. The tubes were shaken for 5 min, the resin allowed to settle and the supernatant aspirated. The resin was next washed 2 times via brief vortexing with 4 ml of water. The washes were aspirated each time after the resin settled. The amino acids were then eluted with 1 ml of 8N NH4OH and 500 μL added to 1.5 ml screwtop microfuge tubes which were dried overnight in a Savant concentrator. To the Savant-dried samples, 50 μL of acetonitrile and 50 μL of N-(tert-butyldimethylsilyl)-N-methyltrifluoroacetamide containing 1% tert-butyldimethylchlorosilane were added. The tubes were capped and heated at 80° C. for 2 hours in a dry-block. After cooling, the samples were spun at 25,000×g for 5 min in a microfuge. The clear reaction supernatants were transferred to 0.1 ml autosampler vials for GC-MS analyses.

For the GC-MS analysis of glutamate, an Agilent bench-top GC-MSD (HP6890/MSD5973) system was autotuned (41, 267 and 599) under PCI conditions with methane as the reagent gas. Next, the reagent gas was switched to ammonia and gas flow optimized via monitoring m/z 52. The GC-MS conditions included: source (150° C.), quadrapole (150° C.), interface (320° C.) and injector (250° C.). The injection port liner was packed with 2% SP-2250 on 100/200 Supelcoport, serving as a pre-column. Aliquots of 1 μL were injected splitless onto the pre-column connected to an HP-5 capillary column (25 m, 0.25 mm i.d. and 0.25 μm thickness) which was held at 120° C. for 1 min followed by 30° C. gradient to 300° C., with carrier gas (He) flow of 1.2 ml/min. The retention times were 5.2 min for GABA and 6.9 min for glutamate. The [MH]+ ions that were monitored were 332 and 338 for endogenous GABA and its internal standard, and 490 and 495 for endogenous glutamate and its internal standard. Five point standard curves were used with each experiment (unlabeled:internal standard=0.25:1, 0.5:1, 1:1, 2:1, 4:1).

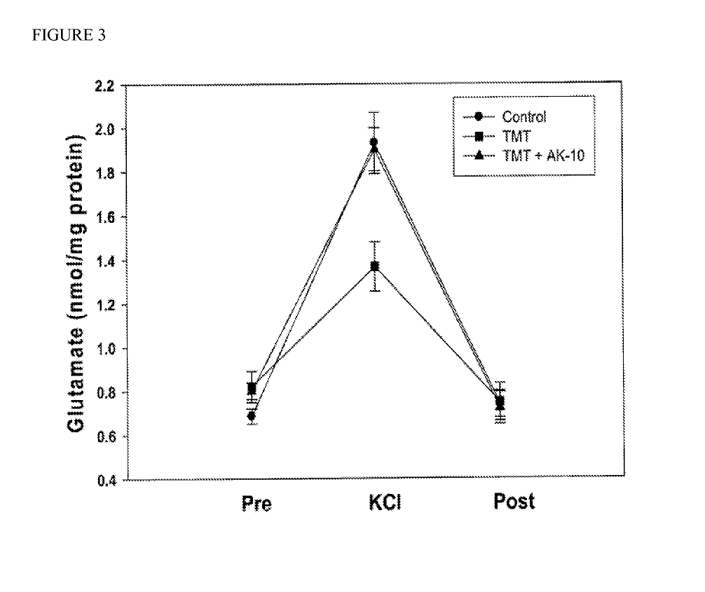

In the rat trimethyltin model of CA3 glutamatergic cell death, the apoptotic cascade is initiated by reactive aldehydes, and protection was 13demonstrated with aldehyde sequestering agents like hydroxylamines and with the hydroxamic acid, AK10 (FIG. 3), but not with antioxidants like ascorbic acid FIG. 3 illustrates prevention of TMT neurotoxicity by treatment with AK-10, administered daily (25 mg/kg, sc) for 17 days, starting 24 hours after TMT (8 mg/kg, sc) treatment. Measurements of KCl-evoked glutamate release from hippocampal slices were used as an index of CA3 neuronal loss. Mean±SEM (N=8-10).

### Example XI

### Methemoglobinemia

Hydroxylamines are efficacious aldehyde sequestering agents but also may induce methemaglobinemia. Methemoglobinemia is the condition caused by the oxidation of hemoglobin into methemoglobin or a deficiency in the ability of the body to reduce methemoglobin to hemoglobin. There are no reports of such effects with hydroxamic acids. lack of methemoglobinemia was validated with AK-10 in the rat using a representative hydroxylamine, N-benzylhydroxylamine, as a positive control (Table 3).

Table 2 sets forth a comparison of the percentage of hemoglobin that is in the form of methemoglobin under control conditions, in the presence of aldehyde sequestering agent, N-enzylhydroxylamine (NBHA) and the hydroxamic acid, AK-10.

TABLE 2

| Treatment | Methemoglobin (%) |
|---|---|
| Control | 2.0 ± 0.2 |
| NBHA (50 mg/kg, sc; 1 hr) | 22.6 ± 3.1 |
| AK-10 (50 mg/kg, sc; 1 hr) | 2.2 ± 0.3 |

Each compound of Formula I that was tested in the above assays was found to be effective for reducing the aldehyde effect and/or was effective in limiting neuronal loss as demonstrated in the TMT assay described above.

It should be understood that various changes and modifications to the present embodiments described herein will be apparent to those skilled in the art. Such changes and modifications can be made without departing from the spirit and scope of the present subject matter and without diminishing its intended advantages. It is therefore intended that such changes and modifications be covered by the appended claims.

What is claimed is:

1. A compound of Formula Ib: 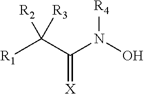
or a pharmaceutically acceptable salt thereof, wherein:
R₁ is selected from the group consisting of C₁₋₆alkyl, C₁₋₆substituted alkyl, C₂₋₆alkenyl, C₂₋₆substituted alkenyl, C₂₋₆alkynyl, C₂₋₆substituted alkynyl, C₃₋₆cycloalkyl, C₃₋₆substituted cycloalkyl, phenyl, cyano, hydroxyl, thiol, sulfonamide, amine, 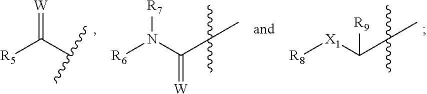
X is oxygen or sulfur;
X₁ is O, S, —S(O)— or —S(O)₂—;
W is oxygen or sulfur;
R₅ is selected from the group consisting of alkoxy, alkyl, substituted alkyl, alkenyl, substituted alkenyl, alkynyl, substituted alkynyl, cycloalkyl, substituted cycloalkyl, cycloalkenyl and substituted cycloalkenyl;
R₆ and R₇ are each independently selected from the group consisting of hydrogen, C₁₋₆alkyl, C₁₋₆substituted alkyl, C₂₋₆alkenyl, C₂₋₆substituted alkenyl, C₂₋₆alkynyl, C₂₋₆substituted alkynyl, C₃₋₆cycloalkyl, C₃₋₆substituted cycloalkyl; or R₆ and R₇ are joined to form an C₃₋₁₀cycloalkyl;
R₈ is selected from the group consisting of hydrogen, C₁₋₆alkyl, C₁₋₆substituted alkyl, C₂₋₆alkenyl, C₂₋₆substituted alkenyl, C₂₋₆alkynyl, C₂₋₆substituted alkynyl, C₃₋₆cycloalkyl, C₃₋₆substituted cycloalkyl; and
R₉ is selected from the group consisting of hydrogen, C₁₋₆alkyl, C₁₋₆substituted alkyl, C₂₋₆alkenyl, C₂₋₆substituted alkenyl, C₂₋₆alkynyl, C₂₋₆substituted alkynyl, C₃₋₆cycloalkyl, C₃₋₆substituted cycloalkyl;
R₂ is selected from the group consisting of hydrogen and C₁₋₆ alkyl;
R₃ is selected from the group consisting of C₁₋₆alkyl-NH—, NH₂—, -alkyl-C(O)—NH—, C₆H₅SO₂NH—, (C₆H₅SO₂)₂N—, C₄H₈N—, and C₅H₁₁NN—; and
R₄ is selected from the group consisting of hydrogen, C₁₋₆alkyl, C₁₋₆substituted alkyl.

2. The compound of claim 1, wherein R₂ is hydrogen.

3. The compound of claim 1, wherein R₄ is H.

4. The compound of claim 1, wherein R₄ is a lower alkyl group.

5. The compound of claim 1, wherein X is oxygen.

6. The compound of claim 1, wherein R₃ is NH₂.

7. The compound of claim 1, wherein R₃ is CH₃—C(O)—NH—.

8. The compound of claim 1, wherein R₁ is iso-butyl.

9. The compound of claim 1, wherein R₁ is propyl.

10. The compound of claim 1, wherein R₂ and R₃ are connected to a chiral center.

11. The compound of claim 1, wherein the compound is selected from the group consisting of: 2-amino-N-hydroxy-4-methylpentamide (Salt TFA), 2-acetoamido-N-hydroxy-4-methylpentamide, 2-amino-N-hydroxypentamide (Salt TFA), 3-amino-N-hydroxy-4-methylpentamide (Salt TFA), 2-amino-N-hydroxypropanamide (Salt TFA), 2-amino-N-hydroxybutanamide (Salt TFA), 2-amino-N-hydroxy-3-methylpentamide (Salt TFA), and 2-amino-N-hydroxy-4-methylpentamide (Salt TFA).

12. A method of treating or preventing a degenerative disease in a mammal comprising administering to said mammal a therapeutically effective amount of a pharmaceutical composition comprising the compound of claim 1.

13. A method of decreasing cell death in a mammal comprising administering to said mammal a therapeutically effective amount of a pharmaceutical composition comprising the compound of claim 1.
